(19) United States
(12) Patent Application Publication
(10) Pub. No.: US 2005/0176937 A1
(43) Pub. Date: Aug. 11, 2005

(54) High throughput screening of potential displacer molecules

(75) Inventors: Steven M. Cramer, Schenectady, NY (US); Kaushal Rege, Troy, NY (US); Jonathan Dordick, Schenectady, NY (US); Nihal Tugcu, Franklin Park, NJ (US)

Correspondence Address: HESLIN ROTHENBERG FARLEY & MESITI PC, 5 COLUMBIA CIRCLE, ALBANY, NY, 12203, US

(73) Assignee: Rensselaer Polytechnic Institute, Troy, NY (US)

(21) Appl. No.: 11/103,293

(22) Filed: Apr. 11, 2005

Related U.S. Application Data
(63) Continuation-in-part of application No. 09/791,317, filed on Feb. 23, 2001, now Pat. No. 6,881,540.
(60) Provisional application No. 60/184,357, filed on Feb. 23, 2000.

(51) Int. Cl. C07H 21/04; C07K 1/10

(52) U.S. Cl. 530/416; 536/25.4

(57) ABSTRACT

A bioproduct may be selectively separated from one or more impurities by means of a displacement chromatography system that includes a solvent, a chromatographic resin and a chemically selective displacer. The method includes: dissolving the bioproduct and the one or more impurities in a solvent; loading the bioproduct and the one or more impurities, in the solvent, on a chromatographic resin; displacing the bioproduct from the chromatographic resin with chemically selective displacer; and retaining the one or more impurities on the chromatographic resin. For this method, the bioproduct and the impurities have similar binding affinity for the chromatographic resin in the absence of the displacer.

### CROSS-REFERENCE TO RELATED APPLICATIONS

This application is a continuation-in-part of U.S. application Ser. No. 09/791,317, filed on Feb. 23, 2001, which claims the benefit of U.S. Provisional Application No. 60/184,357, filed Feb. 23, 2000. The entire disclosure of U.S. application Ser. No. 09/791,317 and U.S. Provisional Application No. 60/184,357 is incorporated herein by reference.

### STATEMENT AS TO RIGHTS UNDER FEDERALLY SPONSORED RESEARCH

This invention was made with support from the National Institutes of Health under Grant No. GM47372-04A2. The United States government may have certain rights in the invention.

### BACKGROUND OF THE INVENTION

Biological macromolecules such as proteins and polynucleotides have become of increasing commercial interest in medicine as pharmaceutical products. Productivity of synthetic processes is frequently limited by purification methods available. Products of biosyntheses are frequently contaminated by structurally similar impurities that must be removed before the product can be used. Chromatographic methods are typically the most effective purification methods, but the physical and chemical similarities between the desired product and the impurities frequently require laborious multiple separations.

Elution chromatography is the mode almost exclusively known and used. However, a chromatographic system may also be operated in a displacement mode, and operation in this mode can have important advantages for purification of bioproducts, particularly on a preparative and/or an industrial scale. Displacement chromatography is distinguishable from elution chromatography both in theory and in practice. In elution chromatography, a solution of the sample to be purified is applied to a stationary phase, commonly in a column. As the mobile phase is passed over the stationary phase, equilibrium is established between the mobile phase and the stationary phase. Depending on its affinity for the stationary phase, the sample species pass along the column at speeds which reflect their affinity relative to other components that may occur in the original sample.

A modification and extension of isocratic elution chromatography is found in step gradient chromatography wherein a series of eluants of varying compositions are passed over the stationary phase.

Displacement chromatography is fundamentally different from elution chromatography (e.g., linear gradient, isocratic or step gradient chromatography). The displacer, having an affinity higher than any of the feed components, competes effectively for adsorption sites on the stationary phase. An important distinction between displacement and desorption is that the displacer front always remains behind the adjacent feed zones in the displacement train, while desorbents (e.g., salt, organic modifiers) move through the feed zones. The implications of this are quite significant in that displacement chromatography can potentially concentrate and purify components from mixtures having low separation factors. In the case of desorption chromatography, however, relatively large separation factors are generally required to give satisfactory resolution.

In displacement chromatography the eluant (i.e., the displacer) has a higher affinity for the stationary phase than do any of the components in the feed. This is in contrast to elution chromatography, where the eluant usually has a lower affinity. The essential operational feature which distinguishes displacement from elution or desorption chromatography is the use of a displacer molecule. In displacement chromatography, the column is first equilibriated with a carrier solvent under conditions in which the components to be separated all have relatively high binding. The feed solution is then introduced into the column following which the displacer is passed through the column. If the displacer and the mobile phase are appropriately chosen, the products exit the column as adjacent square waves zones of highly concentrated pure material in the order of increasing affinity of adsorption. Following the zones of purified components, the displacer emerges from the column. Finally, after the breakthrough of the displacer, the column is regenerated by desorbing the displacer from the stationary phase to allow the next cycle of operation.

Displacement chromatography has some particularly advantageous characteristics for process scale chromatography of biological macromolecules such as proteins. Displacement chromatography can achieve product separation and concentration in a single step unlike elution chromatography which results in product dilution during separation. Since displacement operates in the non-linear region of the equilibrium isotherm, high column loadings are possible. This allows better column utilization than elution chromatography. Finally, displacement can concentrate and purify components from mixtures having low separation factors unlike the relatively large separation factors which are required for satisfactory resolution in desorption chromatography. Displacement is thus a powerful preparative technique that can offer high production rates, resolving power and elevated yields and purity of a desired byproduct.

The main disadvantage of displacement chromatography, and what has limited its application in bioseparations, is the need to identify a displacer molecule for use in each separation. An effective displacer has greater affinity for the stationary phase than the bioproduct to be purified. Additionally, it should cause separation of the bioproduct from impurities on the column. Finally, it should be readily separable from the bioproduct, so that it does not become an impurity itself.

Identification of an effective displacer has been a laborious and tedious task. Displacer candidates are typically screened individually in column experiments using trial and error. While column experiments indicate the exact behavior of displacer molecules in the column, the time required for screening a large number of molecules is a major limitation. A technique for the high throughput screening of potential displacers would enable rapid screening of molecules generated, for example, from a combinatorial library. Screening of a large number of molecules would also provide sufficient data for a predictive QSAR model to actually direct the design of a displacer molecule for a particular bioproduct or a particular stationary phase. This would enable the identification of important properties for a particular interaction or for similar interactions on different stationary phases. Therefore, a need exists for a rapid method for screening a large number of displacer candidates.

### SUMMARY OF THE INVENTION

A rapid method for screening a large number of displacer candidates has been unexpectedly discovered. In this method, a large number of displacer candidates is screened in parallel, enabling extremely rapid assessment of the potential efficacy of each candidate.

In one aspect, then, the present invention relates to a method for screening a plurality of displacer candidates (also called a displacer library, or library of displacers) for efficacy in separating a bioproduct from one or more impurities by means of a displacement chromatography system. The method includes the steps of:
determining the equilibrium concentration of the bioproduct in the mobile phase solvent, in the presence of the stationary phase resin;
for each of the displacer candidates, determining the equilibrium concentration of the bioproduct in the mobile phase solvent, in the presence of both the stationary phase resin and the displacer candidate;
for each of the displacer candidates, determining an amount of the bioproduct displaced from the stationary phase resin; and
rating each displacer candidate according to a relative amount of the bioproduct displaced from the stationary phase resin.

The step of determining the equilibrium concentration of the bioproduct in the mobile phase solvent, in the presence of the stationary phase resin, includes:
equilibrating known amounts of the bioproduct, the one or more impurities, the mobile phase solvent and the stationary phase resin;
performing an analysis of the mobile phase solvent containing the bioproduct, whereby the equilibrium concentration of the bioproduct in the mobile phase solvent, in the presence of the stationary phase resin, is determined.

The step of determining the equilibrium concentration of the bioproduct in the mobile phase solvent, in the presence of both the stationary phase resin and the displacer candidate, includes:
equilibrating a known amount of each displacer candidate, and known amounts of the bioproduct, the one or more impurities, the mobile phase solvent, the stationary phase resin;
performing an analysis of the mobile phase solvent containing the bioproduct; and
whereby the equilibrium concentration of the bioproduct in the mobile phase solvent, in the presence of both the stationary phase resin and a displacer candidate, is determined.

The step of determining an amount of the bioproduct displaced from the stationary phase resin includes:
finding a difference between an equilibrium amount of bioproduct adsorbed on the stationary phase resin in the presence and in the absence of the displacer candidate.

In addition, a displacer library may be screened for chemical selectivity in displacing the desired bioproduct, while leaving the unwanted impurities bound to a stationary phase resin. In this aspect, the method includes the additional steps of:
determining the equilibrium concentration of the one or more impurities in the mobile phase solvent, in the presence of the stationary phase resin;
for each of the displacer candidates, determining the equilibrium concentration of the one or more impurities in the mobile phase solvent, in the presence of both the stationary phase resin and the displacer candidate;
for each of the displacer candidates, determining an amount of the one or more impurities displaced from the stationary phase resin; and
rating each displacer candidate according to a relative amount of the bioproduct displaced relative to the amount of the one or more impurities displaced from the stationary phase resin.

In another aspect, the present invention relates to a kit for use in screening a plurality of displacer candidates for efficacy in separating a bioproduct from one or more impurities by means of a displacement chromatography system comprising a mobile phase solvent and a stationary phase resin. The kit includes a displacer library comprising a plurality of displacer candidates and at least one stationary phase resin. It may additionally include a plurality of sample cells, which may be at least one 96-well microtitre plate. A plurality of stationary phase resins may be included in the kit, as well as a mobile phase solvent.

In yet another aspect, the present invention relates to a method for using a kit comprising a plurality of displacer candidates and a stationary phase resin in screening the plurality of displacer candidates for efficacy in separating a bioproduct from one or more impurities by means of a displacement chromatography system. The method includes the steps of:
combining the stationary phase resin, a mobile phase solvent, and the bioproduct;
determining the equilibrium concentration of the bioproduct in the mobile phase solvent, in the presence of the stationary phase resin;
for each of the displacer candidates, combining the stationary phase resin, a mobile phase solvent, the bioproduct and the displacer candidate;
for each of the displacer candidates, determining the equilibrium concentration of the bioproduct in the mobile phase solvent, in the presence of both the stationary phase resin and the displacer candidate;
for each of the displacer candidates, determining an amount of the bioproduct displaced from the stationary phase resin; and
rating each displacer candidate according to a relative amount of the bioproduct displaced from the stationary phase resin.

In yet another aspect, the present invention relates to method for using a chemically selective displacer in separating a bioproduct from one or more impurities. The method includes the steps of:
dissolving the bioproduct and the one or more impurities in a solvent;
loading the bioproduct and the one or more impurities, in the solvent, on a chromatographic resin;
displacing the bioproduct from the chromatographic resin with the chemically selective displacer.

In the method, the one or more impurities are retained on the chromatographic resin.

The separation may be by means of a displacement chromatography system, where the solvent is a mobile phase solvent, and the chromatographic resin is a stationary phase resin contained in a displacement chromatography column. A chemically selective displacer may be selected using the screening method of the present invention.

### BRIEF DESCRIPTION OF THE DRAWINGS

FIG. 1 is a schematic diagram of a high throughput method for screening displacer candidates according to the present invention.

FIG. 2 is a schematic diagram of a kit according to the present invention containing three 96-well microtitre plates, and its use in automated high throughput screening of a displacer library.

FIGS. 3A-3D are plots of results of high throughput screening of displacer candidates for efficacy in displacing cytochrome C and lysozyme on two different ion exchange resins. Experimental details are described in Example 1.

FIG: 4 Affinity ranking plot for tartrazine, SOS, amyloglucosidase and apoferritin.

FIG. 5: A mixture of apoferritin and amyloglucosidase under linear gradient condition. Column: 50×5 mm I.D. Source 15Q; Buffer A: 20 mM Tris, pH 7.5 and Buffer B: A+600 mM NaCl Gradient slope: 12 mM NaCl/column volume; Flow rate: 0.2 ml/min

FIG. 6: Displacement separation of 24 mg of apoferritin (0.98 mg/ml) and amyloglucosidase (0.75 mg/ml) mixture using SOS as a displacer. Column: 50×5 mm I.D. Source 15Q; Carrier: 20 mM Tris-HCl+30 mM NaCl. pH 7.5; Displacer: 10 mM; Flow rate: 0.2 ml/min

FIG. 7: Displacement separation of 24 mg of apoferritin (0.98 mg/ml) and amyloglucosidase (0.75 mg/ml) mixture using SOS as a displacer. Column: 50×5 mm I.D. Source 15Q; Carrier: 20 mM Tris-HCl+30 mM NaCl, pH 7.5; Displacer: 5 mM; Flow rate: 0.2 ml/min

FIG. 8: Displacement separation of 24 mg of apoferritin (0.98 mg/ml) and amyloglucosidase (0.75 mg/ml) mixture using SOS as a displacer. Column: 50×5 mm I.D. Source 15Q; Carrier: 20 mM Tris-HCl+30 mM NaCl, pH 7.5; Displacer: 3 mM; Flow rate: 0.2 ml/min

FIG. 9: Displacement separation of 24 mg of apoferritin (0.98 mg/ml) and amyloglucosidase (0.75 mg/ml) mixture using tartrazine as a displacer. Column: 50×5 mm I.D. Source 15Q; Carrier: 20 mM Tris-HCl+30 mM NaCl, pH 7.5; Displacer: 10 mM; Flow rate: 0.2 ml/min

### DETAILED DESCRIPTION OF THE INVENTION

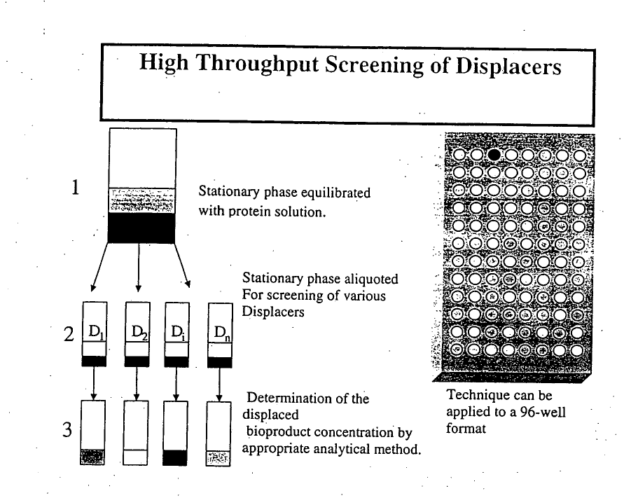

The present invention relates to a method for screening a plurality of displacer candidates for efficacy in separating a bioproduct from one or more impurities, by determining the ability of one or more displacer candidates to displace an adsorbed bioproduct from a stationary phase of a chromatographic system. Bioproducts may be, for example, peptides, proteins, nucleic acids, including olignucleotides, DNA and RNA, polysaccarides, and small molecule natural products and their derivatives. The method may be used with chromatographic systems having different modes of interaction, including ion exchange (IEX), hydrophobic interaction (HIC), and reversed phase (RPLC). Resins useful as a stationary phase for such systems are well known in the art, and are commercially available. Exemplary materials are, for IEX, SP-Sepharose resins available from Amersham Pharmacia, Uppsala, Sweden and Toyopearl ion exchange resins from TosoHaas, Montgomeryville, Pa.; for HIC, the 650M HIC Phenyl, Butyl and Ether series from TosoHaas, and the Phenyl-, Butyl- and Octyl-Sepharose resins from Amersham Pharmacia; and, for RPLC, the Zorbax® series from BTR Separations, Wilmington, Del., Vydac C4, C8 and C18 RPLC columns from Vydac, Hesperia, Calif.; and Octadecyl Silica C18 and Phenyl and Octadecyl resin-based columns from TosoHaas. A diagram showing the method of the present invention schematically is presented as FIG. 1.

In the method of the present invention, the equilibrium concentration of the bioproduct in a particular mobile phase, in the presence of a particular stationary phase, is determined. For each displacer candidate, the equilibrium concentration of the bioproduct in the same mobile phase, in the presence of the stationary phase and the displacer candidate, is also determined. The displacer candidate is rated according to the relative amount of bioproduct displaced from the stationary phase, which is the difference between the concentration of the bioproduct in the presence of the stationary phase, and the concentration in the presence of both the stationary phase and the displacer candidate.

A solution of the bioproduct, either in pure form, or as an impure mixture, in a solvent appropriate for use as a mobile phase, at an appropriate concentration, 2-10 mg/ml, is typically prepared first. Mobile phase solvents are generally specific to the type of stationary phase used, that is, IEX, HIC, or RPLC, and known in the art. Solvent systems for all three modes are generally aqueous, with additives specific to the type of stationary phase used. For HIC, high salt concentrations are used, and for RPLC, organic modifiers, such as acetonitrile and methanol, are employed. For IEX, aqueous buffers are employed, sometimes with added salt.

A known quantity or amount of the bioproduct is equilibrated with a known quantity of the stationary phase material. Equilibration times typically range from less than two hours to about seven hours. The resulting concentration of the bioproduct in the mobile phase is determined by an appropriate analytical method. Analytical methods that are useful for determining concentration of biological molecules such as the bioproducts are well known. For example, spectrophotometric methods may be used to determine the concentration of a protein, by measuring the fraction of light absorbed at a characteristic wavelength, especially in the visible or UV-visible region of the spectrum. Chromatographic methods, including HPLC and capillary zone electrophoresis (CZE), and other analytical methods, including mass spectrometry and NMR, may be used in place of, or in conjunction with, photometric methods. The amount of bioproduct adsorbed on the stationary phase is then calculated by mass balance.

Concentration of the bioproduct in the mobile phase, in the presence of the stationary phase and the displacer candidate, is also determined. The materials may be mixed in any order, although it is preferable that all mixtures should be prepared by the same method. In one embodiment, a known quantity of a displacer candidate is added to a known concentration of the bioproduct in equilibrium with a known quantity of the stationary phase material. Each displacer candidate is typically dissolved in the mobile phase solvent at a known concentration. In another embodiment, after the stationary phase settles by gravity, most of the solvent containing the bioproduct is decanted, and aliquots of the stationary phase are placed in individual cells for analysis. A known amount of a displacer candidate is then added to each. In yet another embodiment, known quantities of each of the bioproduct, the stationary phase and a displacer candidate are mixed and brought to equilibrium. In all of these embodiments, the equilibrium concentration of the bioproduct is determined, generally by the same analytical method used previously. One advantage of the method of the present invention is that the same analytical method may be used to screen all displacer candidates. The amount of bioproduct adsorbed on the stationary phase in the presence of each displacer candidate is calculated by mass balance and compared to the quantity adsorbed without the displacer. The amount of bioproduct displaced from the stationary phase into the mobile phase, relative to the total amount of bioproduct, may be used as a measure of each displacer candidate's ability to preferentially adsorb on the stationary phase, and, thus, to displace the bioproduct. A displacer is considered potentially useful for a separation if it displaces more than about 50% of the bioproduct. The higher the amount of bioproduct displaced, the more efficacious the displacer will be under column conditions. For example, a compound that displaces 90% of the bioproduct is generally effective over a wide range of column conditions. A compound that displaces less than 50% of the bioproduct may require higher concentrations for a separation to be effected under column conditions. High affinity displacers and the correspondingly lower displacer concentrations made possible thereby typically result in higher yields and higher purity in column experiments. Consequently, a rule of thumb that is generally useful is that an effective displacer displaces at least 70% of the bioproduct under the batch screening conditions.

The present invention also relates to a kit for use in screening a plurality of displacer candidates for efficacy in separating a bioproduct from one or more impurities by means of a displacement chromatography system. The kit includes a displacer library composed of a plurality of displacer candidates, and at least one resin useful as a stationary phase of a displacement chromatography system. Compounds useful as displacer candidates have molecular weight less than 10,000 daltons, preferably less than 5000 daltons, and more preferably less than 2000 daltons; are soluble in aqueous solutions at concentration ranging from 1 mM to 100 mM; and contain both hydrophilic and hydrophobic moieties or substituents. Examples of preferred hydrophilic substituents are sulfate, sulfonate, phosphate, phosphonate, carboxylate and quaternary ammonium groups. Examples of preferred hydrophobic substituents are aromatic, substituted aromatic, aliphatic and substituted aliphatic groups. In particular, displacers useful for separation of proteins by ion exchange displacement chromatography are disclosed in U.S. Pat. Nos. 5,478,924 and 5,606,033, specifically, aminoacids, peptides, nucleic acids, antibiotics, amino- or sulfonate-functional dendrimers, aromatic sulfonic or carboxylic acids, and sulfated sugars. Copending U.S. application Ser. No. 09/223,090, filing date Dec. 30, 1998, describes use of low molecular weight, surface-active displacers for purification of proteins in hydrophobic interaction and reversed phase liquid chromatographic systems. The kit may also contain a plurality of sample cells for containing the various displacer solutions, and the mixtures of bioproduct, mobile phase solvent and stationary phase resin, with or without a displacer. In particular, the kit may contain one or more 96-well microtitre plates, optionally, prefilled with any or all of a stationary phase resin, a mobile phase solvent, and members of a displacer library.

The method is particularly advantageous in terms of reducing the amount of time required to evaluate a large number of displacer candidates, because the candidates may be screened simultaneously. Even more advantageously, the screening process may be automated, with one or more of the steps carried out automatically. Automated liquid dispensing equipment and analytical and data handling tools may be used to prepare and analyze samples, and to analyze the data produced. For example, wells of multi-assay plates having a quantity of a stationary phase resin disposed within may be charged with a quantity of the bioproduct in solution. Automatic filling equipment may be utilized for this operation, or the charging operations may be performed by a robotic system. Different displacer candidates may then be added to individual wells. Concentration of the bioproduct in the mobile phase, with and without a displacer, may be determined by automated analytical methods. Devices for automatic analysis of the samples include, for example, vertical-beam photometers, wherein the photometer is able to monitor light absorption of samples, contained in wells of a multi-assay plate, at multiple wavelengths, including the visible or UV-visible region of the spectrum, as well as the near-infrared region of the electromagnetic spectrum. Calculation and tabulation of concentrations and relative amount of the bioproduct displaced may be performed using computer software.

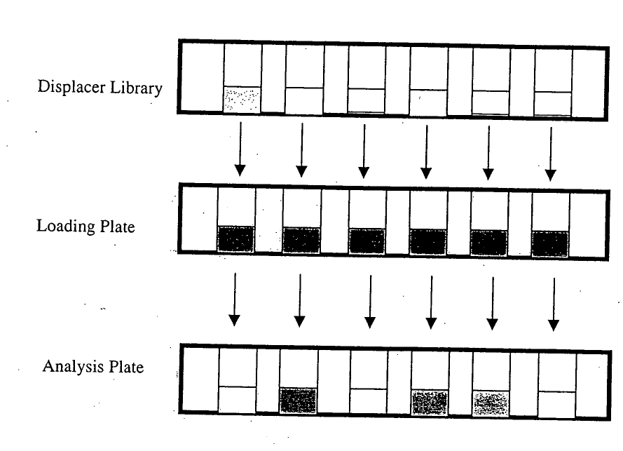

A kit according to the present invention may be used in an automated system to screen a plurality of displacer candidates for efficacy in separating a bioproduct from one or more impurities by means of a displacement chromatography system. For example, three 96-well or -channel microtitre plates or units may be included in a kit. Such a kit is illustrated schematically in FIG. 2. Wells in the top unit may contain different displacers, which may be provided as concentrated solutions in appropriate solvents (e.g., water for ion exchange). Buffers may be utilized in the mobile phase to exploit selectivity based on pH, or ionic strength, if desired. In the middle or the loading unit, wells may contain a stationary phase resin. The bottom, or analysis, unit may be used to collect liquid from the top via a suction mechanism. The top and middle units may be provided with a membrane at the bottom of each well to enable liquid flow from the top to the middle unit, and from the middle unit to the bottom unit.

The bioproduct is equilibrated with the resin in the middle unit, and then the mobile phase may be transferred by suction to the bottom unit for analysis. A vertical-beam spectrophotometer may be used to analyze the samples in the bottom unit, quantifying concentration of the bioproduct by absorbance or fluorescence, for example. The amount of bioproduct adsorbed on the resin may then be calculated by mass balance.

A different displacer is then dispensed into each well by means of a robot or a suction mechanism. After equilibration, the supernatant is transferred to the corresponding wells of the analysis unit aligned below. The liquid in the analysis plate is subjected to an appropriate analysis to determine the amount of bioproduct displaced by individual displacers, thus enabling rapid and parallel screening of displacers for the bioproduct of interest.

The method of the present invention may be used to rapidly screen for displacers on stationary phase resins for different modes of chromatography, including ion-exchange, hydrophobic interaction and reversed-phase. The effect of changes in operating conditions, such as ionic strength, pH, and salt and/or organic modifier composition, may also be rapidly determined. Such a multi-dimensional screening process is particularly advantageous in identifying displacers having chemical selectivity under various conditions, to be used as efficient bioseparation agents.

Data generated from screening a large number of displacer candidates according to the method of the present invention may also be used to develop quantitative structure-efficacy relationship (QSER) models. Such models may then be employed in molecular modeling simulations to design more effective displacers for a particular bioproduct. Candidates identified as potentially effective in simulations may then be screened using the method of the present invention, in a repetitive process, leading to development of very efficient displacers for any particular separation.

In another embodiment, the present invention relates to a method for using a chemically selective displacer in separating a bioproduct from an impurity(ies) by means of a displacement chromatography system. A chemically selective displacer is defined as one that displaces the bioproduct from the stationary phase, but leaves the impurity(ies) bound to the stationary phase, or column, particularly where the bioproduct has affinity for the chromatographic resin similar to that of the impurities in the absence of the displacer. Such a displacer is able to dramatically increase the inherent selectivity of chromatographic systems for a specific biological mixture by altering the affinity of the components of the mixture.

Affinity of the bioproduct and/or the impurities for the resin may be determined by means of an affinity ranking plot using linear SMA parameters (v and K), as described in U.S. Pat. No. 5,478,924 and U.S. Pat. No. 5,606,033, to Cramer, et al., and Gadam and Cramer, Chromatographic 39: 409-418. Where the dynamic affinity lines for the materials are very similar, separation by displacement chromatography is typically difficult. Alternately, retention data obtained with a linear salt gradient may be used to determine whether the materials have similar retention times under gradient conditions, and thus, similar affinity for the resin. The data may be graphed on a linear retention plot and/or adsorption isotherm plot and used to determine relative affinities of the bioproduct and the impurity for the resin. Where the retention times (and affinities) are similar, resolution of the materials is frequently poor. For affinity ranking plots, linear retention plots and adsorption isotherm plots, “similar” means that the lines for the bioproduct and the impurity(ies) are within 6σ of each other, and preferably within 3σ. The chemically selective displacement method differs from displacement chromatographic methods currently practiced in that the compound to be purified, the bioproduct, is displaced from the chromatographic resin, either in batch system, or in a column, while the impurity(ies) are retained on the resin. Such a method has obvious advantages in improving both the level of purification possible and the yield of the chromatographic separation. The chemically selective displacement differs from previously disclosed selective displacement procedures in that difficult separations, that is, of materials having very similar affinity for the chromatographic resin, may be performed using chemically selective displacers, while previous selective displacements that exploited the mass action parameters of the system were successful only with proteins that had different affinities for the resin. See Kundu, A. and Cramer, S. M., Biotechnology and Bioengineering, 56, pages 119-129 (1997).

Steps in the method for using a chemically selective displacer in separating a bioproduct from one or more impurities by means of a displacement chromatography system include dissolving the bioproduct and the one or more impurities in a solvent; loading the bioproduct and the one or more impurities, in the solvent, on a chromatographic resin; and displacing the bioproduct from the chromatographic resin with the chemically selective displacer. The method may be used in a column system where the solvent is a mobile phase solvent, and the chromatographic resin is a stationary phase resin contained in a displacement chromatography column. After the displacer has passed through the column, the impurity(ies) may be retained on the stationary phase resin, or may be eluted in the displacer zone.

The screening method described above may be used to identify chemically selective displacers for a particular separation. In this embodiment of the screening method, the concentration in the mobile phase of both the bioproduct and impurity(ies) is determined, both in the absence and in the presence of the displacer candidate. A rule of thumb that is generally useful is that an effective chemically selective displacer displaces greater than 70% of the bioproduct, and less than 10% of the impurity(ies).

The methods of the present invention may be illustrated with reference to the following examples:

### EXAMPLES

### Example 1

### High Throughput Screening of Displacer Candidates for Protein Separation by Ion Exchange Chromatography

Materials

High Performance S P Sepharose stationary phase material was obtained from Amersham Pharmacia (Uppsala, Sweden). Toyopearl 550 C strong cation exchange resin was obtained from TosoHaas (Montgomeryville, Pa., USA). Phenomenex Jupiter C4 10 μm (250×4.6 mm) column was obtained from Phenomenex, Torrance, Calif., USA.

The potential displacer molecules 2,2 dimethyl-1, 3 propanediamine, 3,3′-diamino-N-methyl-dipropylamine, 5-amino-1,3,3-trimethyl cyclohexane methylamine, butylamine, N,N,N′,N′-tetrakis-(3-aminopropyl)-1,4-butanediamine (DAB(Am)4, polypropyleneaminetetramine dendrimer Gen. 1) diethylenetriamine, hydroxylamine, malonamamidine, malonamide, methylamine, N-methyl-1,3-propanediamine, N,N′-bis-(2-aminoethyl)-1,3-propanediamine, N,N′-bis-(3-aminopropyl)-1,3-propanediamine, N,N′-diethyl-1,3-propanediamine, N,N′N″-trimethyl bis(hexamethylene)triamine, 2-(aminoethyl)-1,3propanediamine, pentaethylene hexamine and tris(2-aminoethyl)amine were p urchased from Aldrich (Milwaukee Wis., USA). Bekanamycin sulfate, butirosin disulfate, histamine, lividomycin sulfate, N-benzoyl-L-arginine ethyl ester, neomycin sulfate, paromomycin sulfate, sodium phosphate (dibasic), sodium phosphate (monobasic), spermidine and were purchased from Sigma (St. Louis, Mo., USA). 1,2 diaminocyclohexane, cyclohexylamine, piperazine hydrochloride were purchased from TCI America, Portland, Oreg., USA.). Pentaerythrityl (dimethylammonium, cyclohexyl methyl (4) iodide (PEDMA Cy I(4)), pentaerythrityl (dimethylammonium, benzyl(6)) chloride (PEDMA BzCl(4)), pentaerythrityl (trimethyl ammonium (4)) (PETMA4), dipentaerythrityl trimethyl ammonium 6 (DPE-TMA(6)) were synthesized at Rensselaer. The proteins, horse heart cytochrome-c and chicken egg lysozyme, were purchased from Sigma (St. Louis, Mo., USA).

Equipment

Absorbance analysis was carried out using a using a Perkin-Elmer Lambda 6.0 UV-Vis Spectrophotometer. (Norwalk, Conn., USA). The chromatographic experiments were carried out using a Waters 600 multisolvent delivery system, a Waters 712 WISP autoinjector and a Waters 484 Uv-Vis absorbance detector controlled by a Millenium chromatography software manager (Waters, Milford, Mass.).

Procedures

High Throughput Screening

The bulk stationary phase was washed with de-ionized water and then the carrier buffer, 50 mM phosphate, pH 6.0 and allowed to equilibrate for 2 hours. After gravity settling of the stationary phase, the supernatant was removed and 3.0 ml of the remaining stationary phase slurry was combined with a solution (36 ml) containing 3 mg/ml of one of the proteins, cytochrome C or lysozyme, in 50 mM phosphate buffer, pH 6.0, at 20° C. The incubation time for the HP Sepharose and Toyopearl 550C materials were 5 and 7 hours, respectively, in order to attain complete equilibrium. After equilibration was complete, the stationary phase was allowed to gravity-settle and the supernatants were removed and the protein content was determined via UV-VIS absorbance analysis (cytochrome C and lysozyme were evaluated at 540 and 300 nm, respectively). The protein adsorbed on the stationary phase was then determined by mass balance.

Aliquots (25 μl) of the remaining slurry containing the stationary phase resin with the bound protein were then added to a 10 mM solution (300 μl) of one of the displacers in 50 mM phosphate buffer, pH 6.0 at 20° C. Displacers were screened in parallel. A total of 33 different displacers were examined for each protein on each stationary phase material. The system was equilibrated for five (HP Sepharose) or seven (Toyopearl 550C) hours and the experiments were carried out in triplicate. Each displacer, displaced a specific amount of protein from the stationary phase material. When the experiment was complete, the supernatant was removed and the protein content was determined via UV-VIS absorbance analysis as described above. (Note: in order to evaluate these small volumes, 200 μl of the supernatant was diluted to 1.2 ml with the buffer). The concentration of protein was determined and the percentage protein displaced was calculated.

Analytical Chromatography

Linear gradient reversed phase chromatography using a Phenomenex Jupiter C4, 10 mm; 250 mm×4.6 mm column was used to evaluate the amount of lysozyme in the supernatant for cases where the displacer interfered with the absorbance assay. A linear gradient of 25% to 90% (v/v) buffer B was carried out in 30 minutes (buffer A: 0.1% (v/v) TFA in de-ionized water; buffer B: 90% (v/v) acetonitrile and 0.1% (v/v) TFA in a de-ionized water). The flow rate was 1 ml/min and the column effluent was monitored at 280 nm.

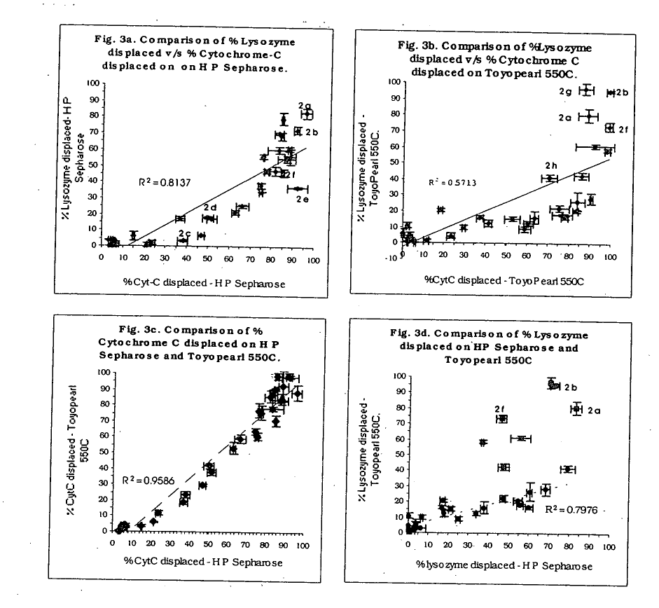

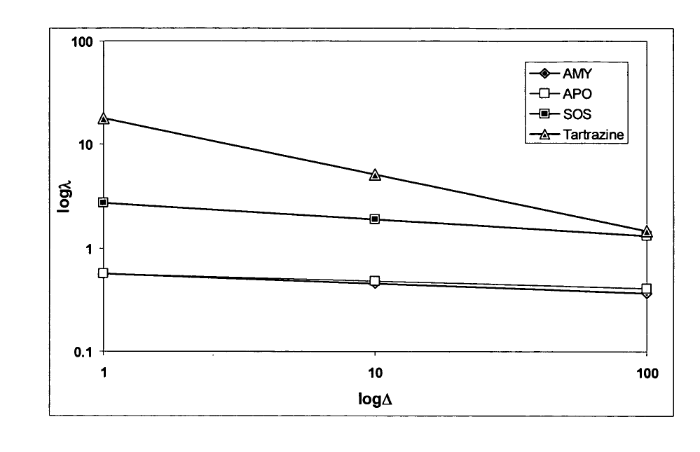

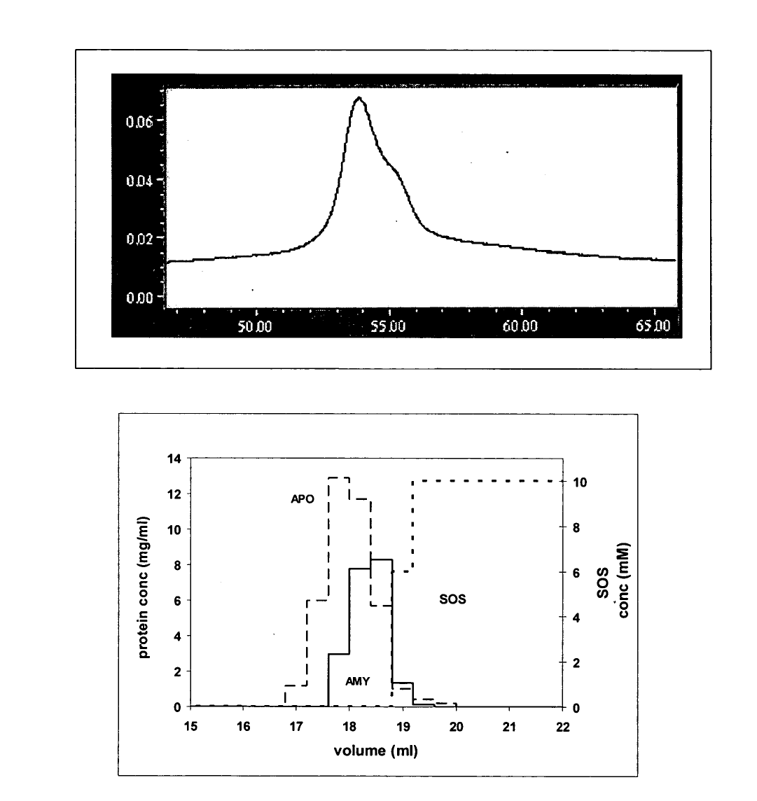

Results obtained with the proteins cytochrome-c and lysozyme on two cation exchange materials (SP-Sepharose HP and Toyopearl 550 C) are presented in Table 1 and FIGS. 3A-3D. Each point on these figures represents a different displacer molecule with the corresponding error bars. The results for the HTS experiments are plotted in the following manner: two proteins on a single resin (FIGS. 3A and B) and one protein on two different resins (FIGS. 3C and D). This is done to enable us to compare the relative efficacy of different displacers for different protein-stationary phase combinations. It is important to note that if the physico-chemical phenomena are similar, then the data should be well correlated.

FIG. 3A shows the data for lysozyme and cytochrome-C on SP Sepharose HP. Values for percent protein displaced were consistently higher for cytochrome C than for lysozyme. This is expected since lysozyme is known to be more strongly retained on the SP Sepharose HP material. However, the data is not particularly well correlated (R²=0.81), indicating that different molecules have various relative efficacies for displacing these two proteins on this resin. Aminoglycosides and highly branched molecules had high efficacy as displacers on Sepharose resin. While, in general, larger molecules with a higher number of charges acted as good displacers for both proteins, an increase in charge did not necessarily imply improved performance as can be seen by comparing the linear molecules N-methyl 1, 3 propanediamine (two charges; 46% cytochrome C displaced) and diethylene triamine (three charges; 35% cytochrome C displaced). In addition, it was observed that cyclic molecules had relatively low affinity for displacing lysozyme as compared to cytochrome C on the SP Sepharose material. For example, PEDMACyI₄, which has four cyclic groups, displaced 36.7% lysozyme as compared to 91.9% for cytochrome C. Clearly, the presence of significant outliers such as PEDMACyI₄ indicates the importance of specific chemistries in determining displacer affinity for a given protein on a specific resin material.

TABLE 1
HTS Data for Cytochrome-C and Lysozyme
on HP Sepharose and Toyopearl 550 C.

| | | | CytC on | Lys on |
|---|---|---|---|---|
| | CytC on HP | Lys on HP | Toyopearl | Toyopearl |
| Displacer | Sepharose | Sepharose | 550° C. | 550° C. |
| Hydroxylamine | 6.23 | 0.43 | 2.25 | 0.00 |
| Histamine | 21.20 | 0.65 | 5.89 | 0.00 |
| Butylamine | 6.72 | 0.00 | 2.16 | 2.07 |
| Malonamide | 3.61 | 0.00 | 0.09 | 4.13 |
| Methylamine | 5.45 | 0.00 | 2.25 | 10.68 |
| 5-amino-1,3,3- | 23.63 | 1.94 | 11.52 | 1.38 |
| trimethyl | | | | |
| cyclohexane | | | | |
| Cyclohexylamine | 6.23 | 2.59 | 3.90 | 3.10 |
| BAEE | 4.97 | 3.24 | 3.12 | 4.48 |
| Diethylene triamine | 38.11 | 3.67 | 23.38 | 4.48 |
| Malonamamidine | 3.12 | 3.67 | 0.17 | 5.86 |
| Piperazine | 14.93 | 6.26 | 3.55 | 3.10 |
| 2,2 dimethyl-1,3 | 46.76 | 6.48 | 29.53 | 9.65 |
| propanediamine | | | | |
| 1,2diamino- | 51.71 | 16.85 | 37.50 | 16.19 |
| cyclohexane | | | | |
| N-methyl 1,3 | 50.06 | 17.71 | 41.65 | 12.40 |
| propanediamine | | | | |
| N,N′-diethyl-1,3- | 36.84 | 17.17 | 18.19 | 20.67 |
| propanediamine | | | | |
| N-2(aminoethyl)- | 62.60 | 21.17 | 52.83 | 15.16 |
| 1,3propanediamine | | | | |
| N,N′bis | 66.09 | 25.05 | 59.06 | 8.96 |
| (2-aminoethyl)-1,3- | | | | |
| propanediamine | | | | |
| Spermidine | 74.94 | 33.69 | 60.53 | 12.40 |
| Tris(2-aminoethyl) | 74.26 | 37.80 | 63.74 | 16.19 |
| amine | | | | |
| Bekanamycin | 75.62 | 55.56 | 76.99 | 18.26 |
| 3,3′-diamino-N- | 77.17 | 47.09 | 75.60 | 22.05 |
| methyl-dipropylamine | | | | |
| Butirosin | 83.10 | 60.19 | 78.03 | 16.54 |
| N,N′N″- | 86.89 | 54.43 | 83.22 | 20.67 |
| trimethyl | | | | |
| bis(hexamethylene) | | | | |
| triamine | | | | |
| Pentaethylene | 87.86 | 60.48 | 84.09 | 26.53 |
| Hexamine | | | | |
| Paromomycin | 84.17 | 68.47 | 90.41 | 28.25 |
| Neomycin | 95.62 | 82.72 | 88.50 | 80.27 |
| PEDMABzCl4 | 84.95 | 45.95 | 98.70 | 73.37 |
| (DAB(Am)4,poly- | 83.10 | 70.03 | 87.21 | 96.76 |
| propylene | | | | |
| aminetetramine | | | | |
| Dendrimer | | | | |
| DPETMA6 | 91.27 | 72.32 | 98.90 | 95.32 |
| PETMA-4 | 88.35 | 55.64 | 92.23 | 60.87 |
| PEDMA CyI4 | 91.95 | 36.72 | 98.12 | 58.57 |
| N,N′bis(3- | 81.45 | 47.09 | 85.91 | 42.38 |
| aminopropyl)-1,3- | | | | |
| propanediamine | | | | |
| Lividomycin | 84.61 | 79.17 | 70.49 | 41.34 |

FIG. 3B shows a comparison of the two proteins on TosoHaas Toyopearl 550 C. As with the Sepharose material, the percent protein displaced values for cytochrome C were generally higher than those for lysozyme.

In FIGS. 3C and 3D the displacer efficacies for a single protein on two different resins are examined. FIG. 3C shows the data for cytochrome C. As seen in the figure, when the data is plotted in this manner, it is relatively well correlated (R2˜0.96). This indicates that the relative efficacy of different molecules for displacing cytochrome C on these two resins is similar. In other words, the stationary phase effects are not pronounced for the displacement of this particular protein.

On the other hand, when the data for lysozyme displacement is examined (FIG. 3D), there is considerable scatter in the plot. PEDMABzCl4, PEDMACyI4, DABPA4 dendrimer generation 1 (Fix) and neomycin were among the outliers showing significantly higher affinities for displacing lysozyme relative to other displacers. These results indicate that it is possible to design displacers which have chemical selectivity for displacing one protein relative to another on a given stationary phase material. In contrast to the results with SP Sepharose, significant differences in affinity were observed for the aminoglycoside family. While lividomycin and neomycin showed relatively high affinities for displacing both proteins; bekanamycin, exhibited relatively low affinity for displacing lysozyme. These results indicate that molecular size does not play a major role in determining the efficacy of these displacers for lysozyme on this material.

Comparison of displacer efficacies for a single protein, cytochrome C, on two different resins shows the relative efficacy of different molecules for displacing cytochrome C on these two resins is similar. In other words, the stationary phase effects are not pronounced for the displacement of this particular protein. On the other hand, when the data for lysozyme displacement is examined, there is considerable scatter. The major outliers are PEDMABzCl4 (2b), PEDMACyI4, DABPA4 dendrimer generation 1, and neomycin. Clearly, these results indicate that displacers can have significant differences in their efficacy for various stationary phase materials. Further, this data indicates that it is possible to design displacers which are particularly efficacious for a given stationary phase material.

### Example 2

### High Throughput Screening of Displacer Candidates for Chemically Selective Separation of Proteins by Ion Exchange Chromatography

A solution of α-chymotrypsinogen A and Ribonuclease A in 50 mM phosphate buffer, pH 6.0 was prepared to a final concentration of 1.5 mg/ml of each protein. HP Sepharose (3 ml) was washed with the buffer and was equilibrated with 36 ml of protein solution above for four hours. The supernatant was removed and analyzed by linear gradient reversed phase chromatography using UV detection at 280 nm. A portion (25 μl) of the stationary phase resin with the two proteins were transferred to vials. As before, 300 μl of a 10 mM displacer solution were added to each aliquot and the system was allowed to equilibrate for 4 hours at 20° C. The supernatant was analyzed by linear gradient reversed phase chromatography using UV detection at 280 nm. The mass of protein in the supernatant was determined and the percent protein displaced was calculated. Results are shown in Table 2.

TABLE 2
HTS data for RNAse A and α-chyA on HP Sepharose.

| | Displacer | α-chyA | RNAse |
|---|---|---|---|
| 1 | BAEE | 1.05 | 3.51 |
| 2 | Malonamamidine | 1.76 | 0.80 |
| 3 | Malonamide | 2.05 | 5.20 |
| 4 | Methylamine | 3.79 | 8.05 |
| 5 | Butylamine | 3.23 | 5.80 |
| 6 | Hydroxylamine | 4.73 | 10.58 |
| 7 | Piperazine | 7.82 | 7.70 |
| 8 | Cyclohexylamine | 18.37 | 3.95 |
| 9 | N,N′-diethyl-1,3 propanediamine | 28.00 | 55.38 |
| 10 | Diethylene Triamine | 28.93 | 57.45 |
| 11 | 2,2-dimethyl 1,3 propanediamine | 34.78 | 60.45 |
| 12 | 1,2 diaminocylclohexane | 41.99 | 50.77 |
| 13 | N methyl 1,3 propanediamine | 54.07 | 64.29 |
| 14 | N(2aminoethylamine)1,3propanediamine | 63.91 | 60.12 |
| 15 | Spermidine | 75.31 | 72.29 |
| 16 | PETMA4 | 83.45 | 77.30 |
| 17 | Pentaethylene Hexamine | 81.14 | 55.30 |
| 18 | 3,3′diamino-N-methyldipropylamine | 87.33 | 55.61 |
| 19 | N,N′,N″-trimethylbis(hexamethylene)triamine | 77.73 | 60.19 |
| 20 | DPETMA6 | 82.96 | 67.81 |
| 21 | Dendrimer | 87.22 | 42.35 |
| 22 | Bekanamycin | 82.04 | 53.11 |
| 23 | Neomycin | 87.25 | 66.70 |
| 24 | Paromomycin | 88.51 | 61.65 |

The table shows that chemically selective displacement is possible using the methods of the present invention.

### Example 3

### Chemically Selective Displacement Chromatography of Apoferritin/Amyloglucosidase Mixture

Experimental Protocol

Materials

Source 15Q (15 μm) strong anion exchange stationary phase material was donated by Amersham Biosciences (Uppsala, Sweden) and the stationary phase was slurry packed into a 50×5 mm I.D. column. TSK-Gel G3000SWXL size exclusion column (300×7.8 mm I.D.) and a TSK-Gel SWXL (40×6 mm I.D.) guard column were donated by TOSOH BIOSEP (Montgomeryville, Pa., USA). Amyloglucosidase and apoferritin were purchased from Sigma (St. Louis, Mo., USA) and ICN Biomedicals, Inc. (Aurora, Ohio, USA), respectively. Sodium chloride and sodium sulfate were purchased from Fisher Scientific (Pittsburgh, Pa., USA). Tris-HCl and Tris-base were purchased from Sigma. Tartrazine and sucrose octasulfate (SOS) were purchased from Aldrich (Milwaukee, Wis., USA) and Toronto Research Chemicals, Inc. (Ontario, Canada), respectively.

Apparatus

Linear gradients were run on a Pharmacia fast protein liquid chromatographic (FPLC) system consisting of two P-500 pumps and a LCC-500 controller (Amersham Biosciences, Uppsala, Sweden). Protein and displacer analysis was carried out using a model 600 multisolvent delivery system, a model 712 WISP autoinjector and a model 996 Photodiode array (PDA) absorbance detector controlled by a Millenium chromatography manager (Waters, Milford, Mass., USA). Absorbance measurements for SOS were carried out using a Lambda 6 UV-VIS spectrophotometer (Perkin Elmer, Wilton, Conn., USA). Fluorescence spectra were collected using a Shimadzu RF-5000 spectrofluorophotometer (Shimadzu Corporation, Kyoto, Japan). Circular dichroism spectra were obtained using a JASCO 720 spectropolarimeter (Japan Spectroscopic Co., Ltd, Japan). SELCON software (http://www.srs.dl.ac.uk/VUV/CD/cpmsd.html) was used for structure analysis.

Procedures

Determination of SMA Parameters for Proteins

The characteristic charge (v and equilibrium constant (K) of the proteins were estimated using isocratic elution chromatography. The isocratic experiments were carried out in 20 mM Tris, pH 7.5 with varying salt concentrations. The resulting capacity factor data was used to provide estimates of the K and y of the proteins. The linear SMA parameters were then used to generate dynamic affinity lines for the proteins. The position of these dynamic affinity lines were used to predict the elution order of the components in a displacement train.

### Determination of SMA Parameters for Displacers

The linear SMA parameters for displacers were obtained using retention times from linear gradient experiments. Once the retention volumes were obtained, using at least two different gradient conditions, the linear SMA parameters, K and v were calculated. Linear gradient experiments were carried out at different total gradient times using buffer A (50 mM Tris, pH 7.5) and buffer B (50 mM Tris, pH 7.5, containing 2.5 M NaCl). Experiments were performed at 0.5 ml/min on Source 15Q. The retention times for tartrazine were obtained by monitoring the UV absorbance at 254 nm. The retention times for SOS were obtained by collecting fractions of the column effluent and assaying for SOS.

### Batch Displacement Experiments

Batch displacement experiments were carried out separately for each protein. The bulk stationary phase (Source 15Q; 2.5 ml) was first washed with deionized water and then the carrier buffer, 20 mM Tris with 30 mM NaCl, pH 7.5 was added, and allowed to equilibrate for 2 hours. After gravity settling of the stationary phase, the supernatant was removed and 30 ml of 1.5 mg/ml amyloglucosidase or apoferritin in the carrier buffer was added and then equilibrated in a shaker for 6 hours at 23° C. The supernatant was analyzed by size exclusion chromatography to determine the protein concentration and the amount adsorbed on the stationary phase was calculated through a mass balance. The supernatant was then removed and 25 μl aliquots of the stationary phase with adsorbed protein were added to separate vials. Aliquots (300 μl) of 10 mM solutions of each displacer in the carrier buffer were then added to each vial, and allowed to equilibrate for 6 hours. After equilibration, the stationary phase was allowed to gravity-settle and the supernatants were removed and analyzed to determine the percentage of protein displaced by each displacer. These experiments were carried out in duplicate.

### Displacement Experiment

For the displacement experiment, a Source 15Q (50×5 mm I.D.) column was initially equilibrated with the carrier buffer 20 mM Tris with 30 mM NaCl, pH 7.5 and then sequentially perfused with feed, displacer and regenerant solutions. The experimental conditions, such as the feed load, flow rate and displacer concentration can be found in the figure legends. Appropriate fractions (400 μl) of the column effluent were collected during the displacement experiments for subsequent analysis of proteins and displacer. The displacement experiment was carried out at a flow rate of 0.2 ml/min and the effluent was monitored at 235 nm. The column was regenerated sequentially with five column volumes of 2 M NaCl and 1 M NaOH with 25 (v/v) % acetonitrile solutions.

### Protein and Displacer Analysis

Protein and tartrazine analysis of the fractions collected during the displacement experiment was performed by size exclusion chromatography using a TSK-Gel G3000SWXL (300×7.8 mm I.D.) with a TSK-Gel SWXL (40×6 mm I.D) guard column with 50 mM phosphate and 100 mM NaCl, pH 6.0. 5 μl samples were injected at a flow rate of 1.0 ml/min and the effluent was monitored at 235 nm. SOS containing fractions were assayed for SOS.

### Measurement of Protein and Displacer Isotherms

All the isotherm measurements were performed in the batch mode. The bulk stationary phase (Source 15Q) was first washed with deionized water and then the carrier buffer (20 mM Tris with 30 mM NaCl, pH 7.5) was added and allowed to equilibrate for 2 hours. After gravity settling of the stationary phase, the supernatant was removed and 25 μl aliquots of the stationary phase were added to separate vials. To measure the single-component isotherms of the proteins, 300 μl of varying concentrations (0.5-15 mg/ml) of each protein in the carrier buffer was added to the individual vials. After equilibration was complete (6 hours), the stationary phases were allowed to gravity-settle and the supernatants were removed and analyzed to determine the concentration of protein in the carrier buffer and on the stationary phase. Multi-component isotherms of both proteins were carried out in the same manner, but with both proteins present in the initial solutions (300 μl) in a 1:1 ratio. Initial protein concentrations varied between 0.25 and 7.5 mg/ml for each protein such that total protein concentration covered the same range as the single component experiments. For multi-component isotherm measurements of each protein in the presence of a displacer, varying concentrations (0.5-15 mg/ml) of protein solutions (300 μl) containing a constant concentration (10 mM) of displacer solution were employed. Multi-component isotherms of both proteins in the presence of 10 mM displacer were measured in the same manner. Again, both proteins were present in the initial solutions (300 μl) in a 1:1 ratio (concentrations varying between 0.25 and 7.5 mg/ml for each protein). For single component isotherm measurement of displacers, varying concentrations (0.25-15 mM for tartrazine and 0.25-10 mM for SOS) of displacer solutions (300 μl) were employed. These experiments were all carried out in triplicate at 23° C.

### HPLC Analysis of Proteins in the Supernatant

Size exclusion chromatography was employed for the analysis of the supernatants in terms of tartrazine, amyloglucosidase and apoferritin content. In these experiments, the carrier buffer was 50 mM phosphate with 100 mM NaCl at pH 6.0. 5 μl of each supernatant solution obtained from the isotherm experiments was injected and the analyses were carried out in duplicate at a flow rate of 1.0 ml/min. The absorbance was monitored between 215 and 280 nm.

### Fluorescence and CD Spectroscopy

Fluorescence spectra of each protein alone (0.5 mg/mL) and in the presence of SOS (10 mM) were obtained. For this purpose, excitation was carried out at 280 nm and emission data were collected between 200 and 600 nm for all cases. CD spectra were collected for the proteins alone and in the presence of tartrazine in the wavelength range of 180 to 260 nm. A band width of 1 nm, cuvettes with 0.02 cm path length and scan speed of 10 nm/min, were employed during these experiments. Three scans were carried out for each solution.

TABLE 3
Linear SMA parameters of proteins and displacers

| | ν | K |
|---|---|---|
| SOS | 6.35 | 597 |
| Tartrazine | 1.85 | 206 |
| amyloglucosidase | 10.36 | 3.04E−3 |
| apoferritin | 13.69 | 4.47E−4 |

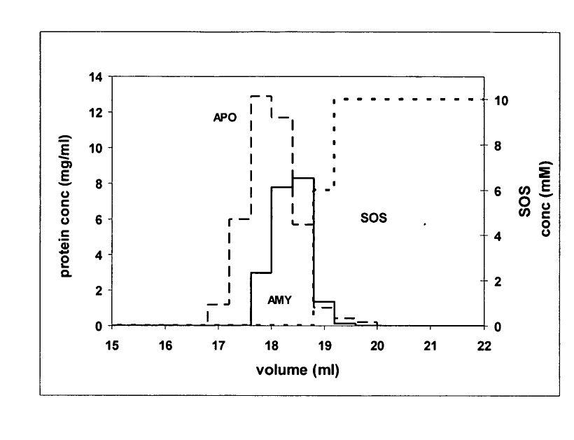

Table 3 shows the linear SMA parameters for the proteins and displacers. FIG. 4 presents the affinity ranking plot where the dynamic affinity lines of tartrazine, SOS, amyloglucosidase and apoferritin are shown. As seen in this figure, the dynamic affinity lines for the two proteins are very similar, indicating that their separation by displacement chromatography will be quite difficult. Further, as shown in FIG. 5, retention data obtained with a linear salt gradient suggested that the two proteins also have very similar retention times under gradient conditions. On this chromatogram, the initial peak represents amyloglucosidase and the shoulder represents apoferritin. Thus, even with a very shallow gradient, the resolution of these two proteins is quite poor.

It can also be seen in FIG. 4 that the dynamic affinity lines for both displacers lie well above the dynamic affinity lines of the proteins, indicating that both displacers should possess sufficient affinity to displace these proteins. In addition, as shown in FIG. 4, the affinity lines do not cross, indicating that the Steric Mass Action model does not anticipate any selectivity changes or any possibility of selective displacement. In contrast to these results, when batch displacement experiments were carried out (Table 2), both tartrazine and SOS exhibited significant selectivity differences with respect to their ability to displace these two proteins.

TABLE 2
Batch displacement results for amyloglucosidase
and apoferritin by using tartrazine and SOS

% Protein Displaced

| Proteins | SOS | Tartrazine |
|---|---|---|
| amyloglucosidase | 40.10 | 59.80 |
| apoferritin | 83.44 | 31.95 |

One significant difference between the affinity ranking plot and batch displacement approaches is that the ranking plots are generated under single-component adsorption conditions, while the batch displacement experiments were run under multi-component conditions (one protein-one displacer). In order to examine this behavior from a different perspective, column displacement experiments were carried out.

### Column Displacement Experiments

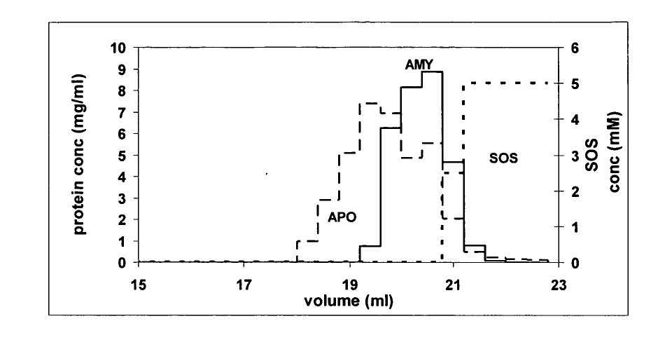

The initial column displacement was run using SOS as the displacer. For this experiment, 10 mM SOS concentration was employed, the same as in the batch displacement experiments. This displacer concentration corresponds to a partition ratio, ≢6 of 24. This quantity is the ratio of displacer concentration on the stationary phase to that in the mobile phase. As seen in FIG. 6, while both proteins could be effectively displaced, amyloglucosidase and apoferritin were not well separated in this column displacement.

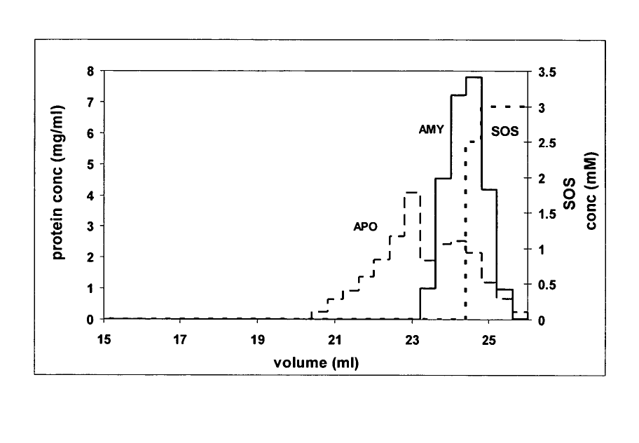

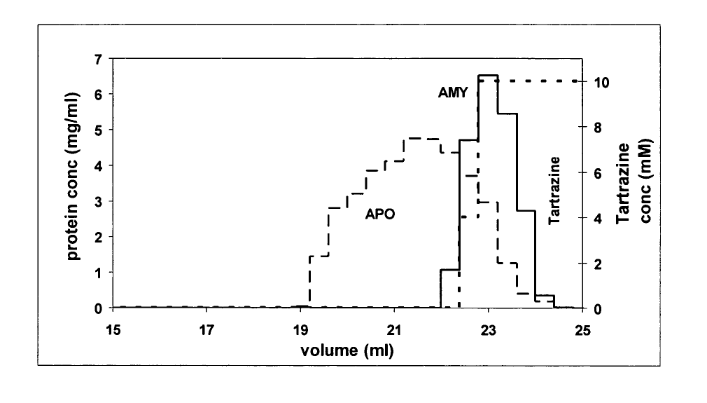

This result agrees qualitatively with the prediction of the dynamic affinity lines (FIG. 4) that both proteins should be readily displaced by SOS, however, the elution order of the proteins were not the same as predicted by the plot. In addition, while the batch experiments indicated that significantly more apoferritin should be displaced when SOS was used as the displacer, the column displacement experiment shows that SOS, at 10 mM, could readily displace both proteins. Finally, due to the difficulty of the separation as evidenced by the proximity of the affinity lines of the two proteins, there was minimal separation of the two proteins in this “traditional” displacement experiment. To evaluate the effect of displacer concentration, experiments with 5 (Δ=38) and 3 mM SOS (Δ=64) were carried out and the results are shown in FIGS. 7 and 8, respectively. While a slight improvement in the resolution can be observed in both displacements due to broadening of the protein zones, the yields of these experiments are still low. In addition, even at these relatively low SOS concentrations, a selective displacement profile could not be obtained.

A column displacement experiment was then carried out using 10 mM tartrazine (Δ=49) as the displacer (FIG. 9). As was shown in Table 2, batch displacement experiments indicated that tartrazine can effectively displace amyloglucosidase but not apoferritin. FIG. 6 shows that this experiment resulted in the selective displacement of apoferritin with amyloglucosidase emerging in the displacer zone. Clearly, selective displacement using tartrazine was not predicted by the SMA model and the dynamic affinity plot. In addition, as seen in the displacements with SOS, apoferritin still elutes from the column first when tartrazine is used as the displacer, in contrast with the batch displacement experiments where significantly more amyloglucosidase was displaced than apoferritin (see Table 2).

These results suggest that there is some discrepancy between the SMA model predictions (FIG. 4) and the observed column displacement experiments. One major difference is the early elution of apoferritin, which could not be predicted with single-component linear SMA parameters. In addition, even though batch displacement experiments had identified both SOS and tartrazine as candidates for selective displacements, only tartrazine actually resulted in a selective displacement of apoferritin over amyloglucosidase. It should be noted that FIG. 4 indicates that the two proteins have extremely similar affinity for the resin AND that, according to conventional wisdom, both of these displacers should be able to displace both proteins under all possible operating conditions. In contrast, FIG. 6 demonstrates that under column conditions, a selective displacement occurs in sharp contrast to what would be expected from the art.

1. A method for selectively separating a bioproduct from one or more impurities by means of a displacement chromatography system comprising a solvent, a chromatographic resin and a chemically selective displacer, said method comprising:
dissolving the bioproduct and the one or more impurities in a solvent;
loading the bioproduct and the one or more impurities, in the solvent, on a chromatographic resin;
displacing the bioproduct from the chromatographic resin with chemically selective displacer; and
retaining the one or more impurities on the chromatographic resin;
wherein the bioproduct and the one or more impurities have similar binding affinity for the chromatographic resin in the absence of the displacer.

2. A method according to claim 1, wherein said solvent is a mobile phase solvent, and said chromatographic resin is a stationary phase resin contained in a chromatography column.

3. A method according to claim 1, wherein said chemically selective displacer is identified by screening a plurality of displacer candidates for efficacy in separating the bioproduct from the one or more impurities in a plurality of extra-column, high throughput, parallel screening batches.

4. A method according to claim 1, wherein molecular weight of said chemically selective displacer is less than 10,000 daltons.

5. A method according to claim 1, wherein molecular weight of said chemically selective displacer is less than 5000 daltons.

6. A method according to claim 1, wherein molecular weight of said chemically selective displacer is less than 2000 daltons.

7. A method according to claim 1, wherein said chemically selective displacer contains both hydrophilic and hydrophobic substituents.

8. A method according to claim 1, wherein the chemically selective displacer contains hydrophilic substituents selected from sulfate, sulfonate, phosphate, phosphonate, carboxylate and quaternary ammonium groups.

9. A method according to claim 1, wherein the chemically selective displacer contains hydrophobic substituents selected from aromatic, substituted aromatic, aliphatic and substituted aliphatic groups.

10. A method according to claim 1, wherein the chromatographic resin is an ion exchange resin.

11. A method according to claim 1, the chemically selective displacer is selected from aminoacids, peptides, nucleic acids, antibiotics, amino-functional dendrimers, sulfonate-functional dendrimers, aromatic sulfonic acids, aromatic carboxylic acids, and sulfated sugars.

12. A method according to claim 1, wherein the chromatographic system is a hydrophobic interaction chromatographic system.

13. A method according to claim 1, wherein the chromatographic system is a reversed phase liquid chromatographic system.

14. A method according to claim 1, wherein the bioproduct is a protein.

15. A method according to claim 1, wherein greater than 70% of the bioproduct is displaced and greater than 90% of the one or more impurities is retained.

16. A kit for use in screening a plurality of displacer candidates for efficacy in separating a bioproduct from one or more impurities by means of a displacement chromatography system comprising a mobile phase solvent and a stationary phase resin, said kit comprising a displacer library comprising a plurality of displacer candidates and at least one stationary phase resin.

17. A kit according to claim 16, additionally comprising a plurality of sample cells.

18. A kit according to claim 17, wherein said plurality of sample cells comprises at least one 96-well microtitre plate.

19. A kit according to claim 16, wherein said at least one stationary phase resin comprises a plurality of stationary phase resins.

20. A kit according to claim 16, additionally comprising a mobile phase solvent.
